(19) United States
(12) Patent Application Publication
(10) Pub. No.: US 2008/0200843 A1
(43) Pub. Date: Aug. 21, 2008

(54) Method and Apparatus for Measurement of Human Tissue Properties in Vivo

(75) Inventors: Robert L. Williams, Athens, OH (US); John N. Howell, Athens, OH (US); Robert R. Conatser, Athens, OH (US); David H. Noyes, Millfield, OH (US); Janet M. Burns, Athens, OH (US)

Correspondence Address: CALFEE HALTER & GRISWOLD, LLP, 800 SUPERIOR AVENUE, SUITE 1400, CLEVELAND, OH, 44114, US

(73) Assignee: OHIO UNIVERSTIY, Athens, OH (US)

(21) Appl. No.: 12/063,075

(22) PCT Filed: Aug. 9, 2006
(86) PCT No.: PCT/US06/31083

§ 371 Date: Feb. 6, 2008

Related U.S. Application Data
(60) Provisional application No. 60/706,739, filed on Aug. 9, 2005.

(51) Int. Cl. A61B 5/00

(52) U.S. Cl. 600/587

(57) ABSTRACT

A method and apparatus that applies a predetermined force function to the surface of a test subject with a probe and measures the displacement of the probe as a function of applied force facilitates measurement of tissue properties accurately and quickly, in vivo, in a non-invasive manner. A haptic device may be used to apply the force function to the test subject according to a preprogrammed force function and to measure the resulting tissue displacement.

### TECHNICAL FIELD

The invention relates generally to the field of the measurement of human tissue properties and more particularly to the field of in vivo human tissue measurement.

### BACKGROUND

In the past, the most common form of human tissue properties measurement has been with cadaver-based measurements. Whether the deceased subject was embalmed or not, this method is inadequate for realistically simulating the behavior of live human tissue.

The simulation group of CIMIT has been measuring the properties of organs for virtual physics-based surgery simulation by removing subject organs and exposing them to mechanical displacements and observing the responding forces and displacements. For in vivo measurements there are currently two options: a non-invasive, image-based method examining the strain fields within living tissues subject to force fields; and invasive methods based on measuring the force-displacement responses of tissues. For invasive methods, laparoscopic methods are common, generally using pigs due to their similarity to human organs. Wang et al. have developed a sensor for in vivo analysis of multiple-layer buttocks soft tissue analysis to help identify persons subject to pressure ulcers. Edsberg et al. experimented with human skin in vitro via uniaxial tensile testing, reporting the first compressive-pre-load-induced strain softening of a biological material. EnduraTEC is involved with all kinds of biological and bioengineering materials studies: teeth, vocal cords, cartilage, artificial heart valves and stents, liver, FEA orthotic heel model, and spinal disc implants. However, most of their materials are engineered; of the biological tissue studies, all are in vitro or in animal subjects (pigs and cows).

U.S. Pat. No. 4,132,224 to Randolph describes a durometer that can be used to determine the surface hardness of human tissue for dental and medical use in identifying edema, swelling, puffiness, and distension. U.S. Pat. No. 5,373,730 to Kovacevic concerns a hand-held device for skin compliance measurements in medical and dental cases where tissues must bear loads or swell after treatment. Neurogenic Technologies, Inc., has developed the Myotonometer®, a hand-held measurement system to quickly assess relative muscle stiffness, tone, compliance, strength, and spasm.

### SUMMARY

A method and apparatus for applying a predetermined force function to the surface of a test subject with a probe and measuring the displacement of the probe as a function of the applied force facilitates measurement of tissue properties accurately and quickly, in vivo, in a non-invasive manner.

Accordingly, a method is provided that determines a model of a compliance related property of a target tissue of an animal or human test subject. A force function to be applied to the test subject is determined. The force function can include any progression of force levels and can be, for example, a series of force steps of increasing force and constant duration or a sinusoidal force. A force is applied according to the force function on an exterior surface of the test subject that overlays the target tissue. A displacement of the probe is measured during application of the force, such as, for example, at the end of the duration of each force step in the series of force steps or at other appropriate times. A compliance function is formed that correlates the measured displacement to the applied force.

It may be advantageous during performance of the method to position the probe such that the force is applied in a direction normal to the exterior surface of the test subject. The compliance function may be formed by determining a best fit line that describes the displacement as a function of applied force and selecting the slope of the line to model a compliance of the target tissue. The compliance function may be formed by determining a best fit curve that describes the displacement as a function of applied force and selecting the slope of the curve at each applied force interval to model a compliance of the target tissue.

The compliance related property being modeled may be a viscous damping coefficient of the tissue, in which case, a rate of change of displacement of the probe as a function of time is determined and the compliance function is formed using a model that correlates the rate of change of displacement to the force function. For example, the compliance function may be a first order linear model that expresses force as the sum of the product of the viscous damping coefficient and the first derivative of the displacement as a function of time and the product of a static spring coefficient and the displacement as a function of time. In some instances the compliance function is a second order linear model that expresses a change in displacement in response to an input force as a function of the first and second derivatives of the displacement as a function of time; a damping ratio, the natural frequency, and the displacement as a function of time.

In some circumstances is it advantageous to monitor EMG signals from sensors connected to the test subject and to measure displacement at predetermined EMG levels. To track therapeutic progress of the test subject, the tissue measurement method may be repeated periodically on a the subject to determine changes in tissue condition.

An apparatus is provided that determines a model of a compliance related property of a target tissue in a test subject. The apparatus includes a probe that is adapted to contact and apply force to an exterior surface of the test subject, a probe driver, a compliance modeler, and a compliance modeling interface. The probe driver is adapted to receive a force function and cause the probe to apply according to the force function and to measure a displacement of the probe during application of the force. The compliance modeler is in communication with the probe driver and forms a compliance function that correlates measured displacement to the applied force. The compliance modeling interface is configured to accept a force function from a user and transmit the force function to the probe driver; receive displacement data from the probe driver; and transmit the displacement data and data indicative of the force applied to the subject to the compliance modeler. In the described embodiment, the probe driver is a haptic device that applies forces to the subject according to the force function received from the compliance modeling interface. In some instances, the apparatus also includes an EMG monitor that monitors and displays EMG level in the target tissue to test subject. In some cases the probe driver may be configured to accept a value for a desired contact angle with the test subject from the compliance modeling interface. It may be advantageous to include a user interface that provides an interface for a user to input a desired force function and displays the resulting compliance function to the user.

### BRIEF DESCRIPTION OF THE DRAWINGS

FIG. 1 is a schematic block diagram of a tissue compliance modeling system constructed in accordance with an embodiment of the present invention;

FIG. 2 is a flowchart outlining a method of modeling tissue compliance according to an embodiment of the present invention;

FIG. 3 is a perspective view of a haptic device used to apply force and measure tissue displacement according to an embodiment of the present invention;

FIG. 4 is an example of a compliance curve that is generated by an embodiment of the present invention;

FIGS. 5 and 6 are examples of tissue displacement data generated by an embodiment of the present invention and plotted as a function of time; and

FIGS. 7-10 are example presentations of tissue compliance model results according to an embodiment of the present invention.

### DETAILED DESCRIPTION

The tissue properties required for constructing the virtual human models mentioned above are generally 3D compliance, as defined below. The inverse of compliance, stiffness, is also of interest. The definitions below are general; they may be adapted for specific XYZ Cartesian directions, one by one, to obtain the general 3D compliance (and stiffness) properties. Units given below are millimeters (mm) for displacement and Newtons (N) for force. Human tissue is generally nonlinear, non-homogeneous, and non-isotropic, greatly complicating the properties measurement compared to common engineering materials.

$$\begin{matrix}Compliance = \frac{Displacement}{Force} {Δ}_{=} \frac{mm}{N} \\ Stiffness = \frac{Force}{Displacement} {Δ}_{=} \frac{N}{mm}\end{matrix} \qquad ( 1 )$$

Accordingly, data from the compliance modeling techniques and devices described herein can be used in many applications, for example: 1. To provide realistic haptic properties for construction of virtual human models; 2. To measure the compliance of Fibromyalgia patients' at tenderpoints to study and improve treatment; and 3. To measure human body properties for a range of subjects (varying age, gender, and body type) to support industrial and consumer products design.

### System Overview

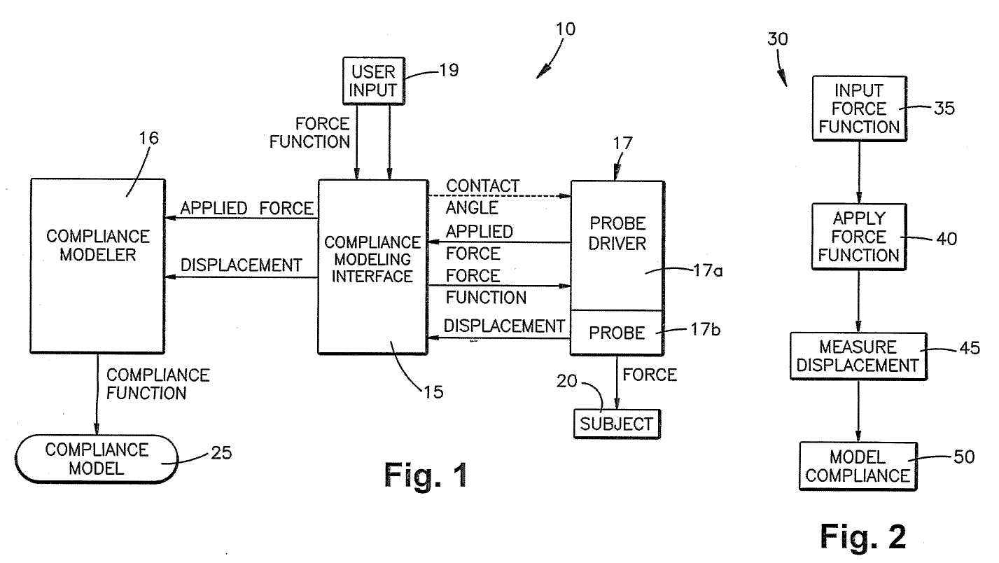

FIG. 1 is a schematic block diagram of a compliance modeling system 10 that includes a compliance modeling interface 15, and a force applicator 17 including a probe 17b and a probe driver 17a. A user input 19, such as a PC executing an input routine, may be used to accept desired parameters from a user and to display the compliance results to the user via the compliance modeler interface 15. For example, the user may input a desired force function to be applied by the probe 17b to the subject. The force applicator 17 is configured to contact and apply the force specified by the force function to the test subject 20, such as a human or animal.

Because the compliance model may be used to construct virtual human models that will be acted on by the hands of medical personnel, it is advantageous that the probe 17b be similar in size, shape, and compliance to a human finger, however, other probe configurations are contemplated within the scope of the described system. The probe 17b is controlled by the probe driver 17a according to the force function, which is received from the compliance modeling interface 15, and that varies as a function of time. The probe driver 17a causes the probe 17b to apply force to the subject 20 according to the force function. While applying force to the subject, the probe 17b measures its own displacement from an initial contact point that results from the application of the force. The displacement data is sent to the compliance modeling interface 15. The compliance modeling interface 15 receives the displacement data and a record of the applied force, which it configures for input to a compliance modeler. The compliance modeler determines a compliance model 25 of the test subject 20 based on the force and displacement data. The compliance modeling interface is configure to receive a desired force function to be applied to the subject.

The compliance modeling interface 15 includes hardware in the form of input and output ports as well as computer processing capability for storing and executing software. The compliance modeling interface 15 includes input connections for receiving the desired force function from the user input and software and/or hardware that configures the input desired force function into instructions and/or signals for output to the force applicator 17. The compliance modeling interface also includes input connections for receiving displacement and applied force data from the force applicator 17. The compliance modeling interface 15 includes software and/or hardware that configures the input applied force and displacement data to be output to the compliance modeler 16.

The compliance modeling interface may also provide positional information to the probe driver in the form of a desired contact angle. The desired contact angle can be set by the probe driver in response to the input contact angle or, alternatively, the contact angle may be set manually. As with the force function, the contact angle can be provided to the compliance modeling interface through the user input 19.

The compliance modeler 16 and/or user input 19 may be implemented in the form of computer-executable instructions or one or more software applications stored on a computing device capable of executing the instructions or software, such as a PC that includes a display. The compliance model may, for example, be presented in the form of one or more graphs that depict or predict tissue displacement as a function of applied force as well as mathematical equations that describe the relationship between displacement and applied force.

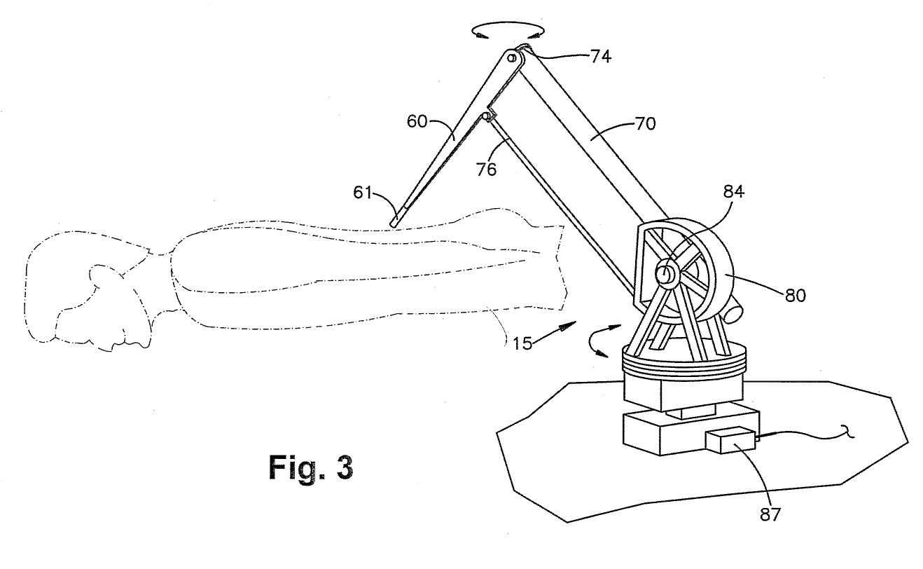

The compliance modeling system 10 can be operated according a method 30 that is outlined in flowchart form in FIG. 2. At 35 a force function is input to the probe driver (17a, FIG. 1) by the compliance modeling interface (15, FIG. 1). The force function may have been received from the user input (19, FIG. 1) or may be stored in the probe driver for repeated use. At 40, the probe driver causes the probe (17b, FIG. 1) to apply force according to the force function, such as a series of discrete force levels, a sinusoidal force, or any other pattern of forces, to the subject. At 45, the probe sends signals indicative of displacement measurements and applied forces to the compliance modeling interface which in turn configures and sends data representing the displacement and applied force to the compliance modeler. At 50, the compliance modeler outputs a compliance model.

### Haptic Device

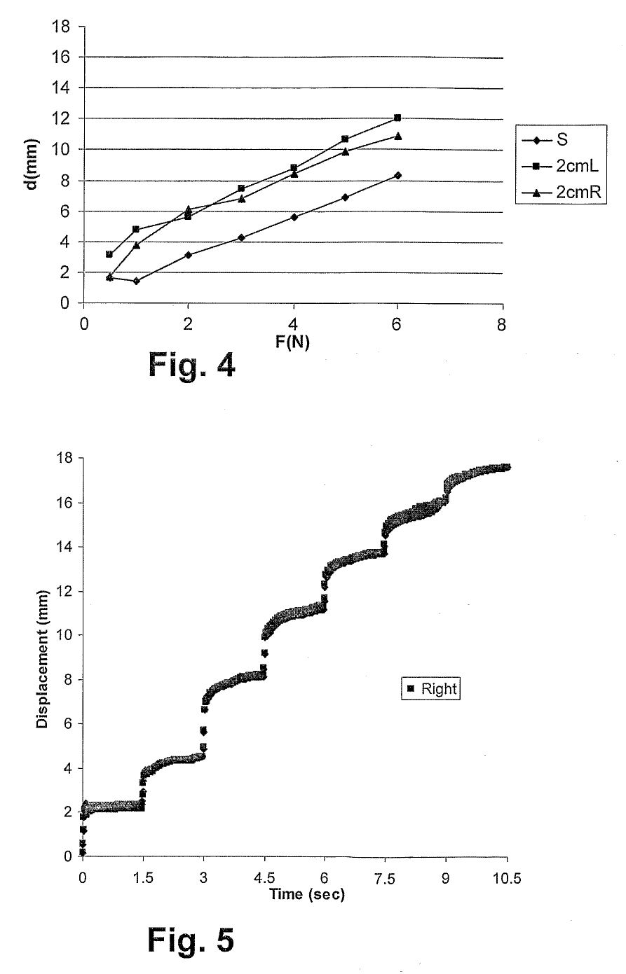

FIG. 3 shows an example force applicator 17 that is constructed from a stock haptic device available from SensAble Technologies, Inc and sold as the PHANToM®3.0. The exact specifications of the device can be obtained from product literature, and will be briefly summarized here. The device is capable of exerting forces in the x, y, and z directions and measuring displacements in the x, y, z directions. It can be modified by the manufacturer to measure roll, pitch, yaw angles. The device has a nominal position resolution of 0.02 mm, a maximum exertable force of 22 N, a continuous exertable force of 3 N, a stiffness of 1 N/mm, a backdrive function of 0.2 N, and an inertia of less than 150 g. The haptic device 17 includes a first arm 60 pivotally coupled to a second arm 70 about a pivot point 74. The first arm has at its distal end a compliant probe 61 shaped to approximate a fingertip. The position of the first arm relative to the second arm is controlled by control rod 76. The haptic device includes a driving mechanism 80 that rotates the second arm 70 about a pivot point 84 to apply the force function to the subject via the probe. Displacement and force data are output through a port module 87 to the compliance modeling interface 15 and force function and other operating parameters are input to the haptic device through the port module.

For the purposes of this description, the performance of vivo human body compliance measurement methods include exerting step inputs of force via the PHANToM® 3.0 in steps of 0.5, 1, 2, 3, 4, 5, and 6 N. A first calibration technique prior to each day of measurements is to command the PHANToM® 3.0 to exert these levels of force on an external force transducer and ensure that the desired force levels are achieved in reality. The device produces very good results with all such static force calibrations, within hundredths or even thousandths of a Newton at all force levels, in various positions. The compliance of the PHANToM® 3.0 itself is calibrated because the device is not rigid. It has been observed that the device has average compliance values of 0.3748 mm/N for one of the devices referred to as “left” and 0.4417 mm/N for the other of the PHANToM® 3.0s, referred to as “right.”

If the PHANToM® 3.0 is significantly stiffer than the human tissue measured, there will be little error due to this internal compliance. Assuming a simple series spring model with the applied force acting through the PHANToM® 3.0 stiffness K_{P} in series with the human tissue stiffness K_{H}, the equivalent spring stiffness K_{EQ} is

$${K}_{EQ} = \frac{{K}_{P} {K}_{H}}{{K}_{P} + {K}_{H}} \qquad ( 2 )$$

That is, the overall stiffness is less than either component stiffness. Conversely, the overall equivalent compliance is

C_{EQ}=C_{P}+C_{H} (3)

and so the human tissue compliance is found from C_{H}=C_{EQ}−C_{P}, where the equivalent compliance C_{EQ} is measured (see methods below) and the PHANToM® 3.0 compliances C_{P} were stated above, for our left and right PHANToM® 3.0s.

The described in vivo human tissue compliance measurement technique has been used for the human back, the abdomen, and tenderpoint measurements for Fibromyalgia studies.

### Human Tissue Compliance Measurement

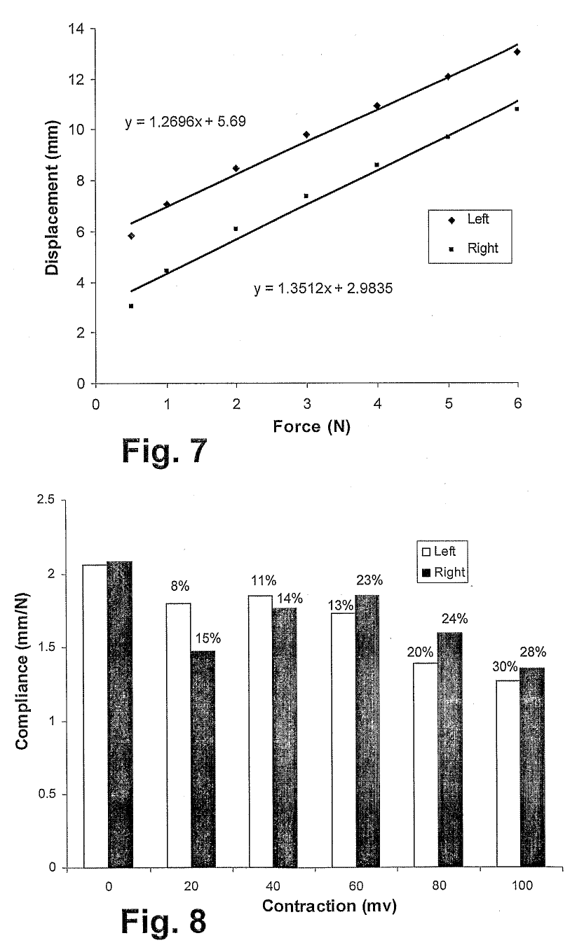

During back compliance measurement, the subject is prone (though many subject and measurement tool orientation schemes are possible) and the surface properties of the back are measured at vertebra T7. The seated operator has placed the tip of the PHANToM® 3.0 commercial haptic device, fitted with a rounded tip the size of an average adult finger pad, at the desired location. The haptic device is commanded to exert seven increasing step levels of force (0.5, 1, 2, 3, 4, 5, and 6 N exerted every 1.5 see). For each force, the displacement into the back is measured by haptic device encoders and forward pose kinematics and output by the system to the compliance modeler. For static compliance measurements a single displacement value is taken near the end of each 1.5 sec application time, prior to increasing the input force in another step and repeating the process, while the subject holds their breath. The resulting displacement data is plotted on the dependent axis vs. the force on the independent axis. If the result is linear, the slope of this line is the compliance into the back at this point on the subject. If the result is nonlinear, the compliance changes, defined by the slope of the curve at each point. The compliances at this point in the remaining Cartesian directions (in the plane of the back, normal to the direction being measured in FIG. 5) are measured in a similar manner.

The measurement tool (PHANToM® 3.0) is accurate and calibrated to real-world units mm and N. However, there are a few challenges which must be overcome to ensure accurate and realistic compliance results. The system is sensitive enough to pick up the human heartbeat. Breathing can interfere with the abdominal properties measurement. Therefore, the subject is asked to take three deep breaths in succession, then take half a breath and hold it in, closing the glottis and relaxing all muscles. Then the force is applied and the corresponding displacement recorded. The haptic device is instructed to exert the seven force levels every 1.5 sec, and the data is analyzed for one breath cycle.

Since human backs are 3D surfaces and not flat planes, the PHANToM® is instructed to exert force into the normal direction of the back at each measurement point, rather than only along a global vertical direction that is not necessarily perpendicular to the back. At each measurement point of interest an angle measuring device is used to ascertain the angles (in two orthogonal directions) of the surface relative to absolute vertical. Then these numbers are entered into the user input and the forces are exerted in the desired direction, normal to the human back. The measurement process could also be automated by utilizing the automatic angle-measuring capability from the manufacturer.

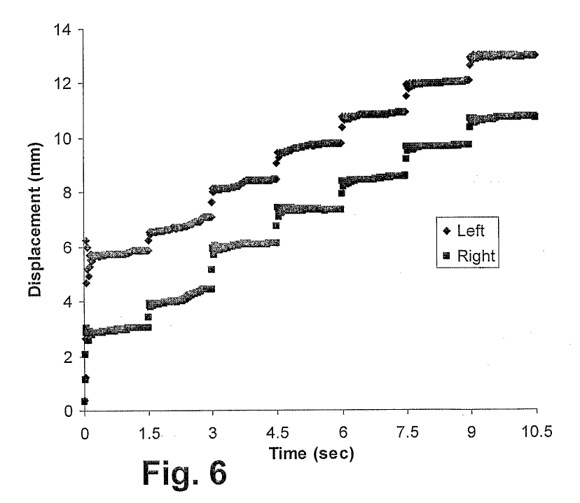

FIGS. 4 and 5 are examples of data from pilot studies with the in vivo measurement of human back compliance properties using the commercial haptic device. FIG. 4 shows the compliance curve (dependent measurement displacement d, mm, vs. independent applied force F, N) for vertebra T10, including the center (S, which stands for spinous process), 2 cm left of center, and 2 cm right of center. The graph is for compliance normal (into) to the subject back. As can be seen in FIG. 4, compliance is about 1.4 mm/N over the spinous process as well as over the ribs, both being boney. For reasons that are not clear the compliances in the thoracic region appear more linear than in the lumbar region. FIG. 5 shows the recorded displacement data upon which the graph in FIG. 4 is based.

The static compliance and stiffness definitions above to include a component of time, with Mobility and Impedance:

$$\begin{matrix}Mobility = \frac{Velocity}{Force} {Δ}_{=} \frac{mm}{Ns} \\ Impedeance = \frac{Force}{Velocity} {Δ}_{=} \frac{mm}{mm}\end{matrix} \qquad ( 4 )$$

In parallel with the static compliance measurements discussed above, dynamic measurements of the human abdomen and lumbar region can be made. The same discussion from the static measurements applies, with additional considerations discussed in this section. This can lead to the experimental determination of viscoelastic models for the dynamic compliance of the range of human abdomens under consideration.

For static measurements a given step change in force is applied while the displacement into the tissue is measured, both with the PHANToMS 3.0 haptic devices. Currently, each force level is held for 1.5 sec and the displacements are measured in mm (see FIG. 5). For static compliance a single displacement value is taken near the end of the 1.5 sec application time, prior to increasing the input force in another step and repeating the process, while the subject holds their breath. The step levels of input, forces are 0.5, 1, 2, 3, 4, 5, and 6 N in FIG. 5.

For simple dynamic measurements the same procedure is followed, but all of the data over time is used rather than taking one final displacement value for each step input force level. The time level is increased to about 5 sec for each measurement to ensure all applicable dynamic results are captured (in FIG. 5 it can be seen that 1.5 see is not sufficient, even for the relatively stiff cervical vertebrae area, especially for higher step input force levels).

From preliminary dynamic measurements (see FIG. 5, from a static compliance measurement run with 1.5 sec time steps) it appears that a first-order system will capture the dynamic human tissue behavior adequately. Thus a linear viscoelastic model is possible such that cx(t)+kx(t)=f(t), where x(t) is the displacement, x(t) is the velocity, f(t) is the applied input force magnitude, and c and k are the lumped, constant viscoelastic parameters (viscous damping and spring stiffness coefficients, respectively) for each point of measurement. From the experimental data (displacement vs. time) the time constant τ can be determined. After three time constants (3τ), the displacement rises to within 5% of the final step change displacement value. Thus, by measuring the time constant and taking the dynamic spring stiffness to be the static spring stiffness, the viscous damping coefficient can be determined:

$$\begin{matrix}τ = \frac{c}{k} & c = k τ\end{matrix} \qquad ( 5 )$$

If the first-order model is insufficient in some cases, the experimental data can be fitted for a standard second-order linear system model: \ddot{x}(t)+2ξωₙ\dot{x}(t)+ωₙ²x(t)=ωₙ²u(t) where ξ is the dimensionless damping ratio, ωₙ is the natural frequency, and u(t) is now the displacement step change caused by the input step force. These generic parameters are related to the dynamic mechanical tissue properties by:

$$\begin{matrix}ξ = \frac{c}{2 \sqrt{km}} & {ω}_{n} = \sqrt{\frac{k}{m}}\end{matrix} \qquad ( 6 )$$

Also, f(t)=A sin(ωt) can be used as a sinusoidal force input, in place of the proposed step changes in input force. By varying the driving force frequency ω, the frequency response of each desired point on the human can be measured.

To measure abdominal compliance, each subject's abdomen is measured every 20 degrees (from a top view); at each of these measurement planes there will be three planes for measurement, spaced evenly vertically to cover the anatomy of interest. At each measurement location the seven step forces (0.5, 1, 2, 3, 4, 5, and 6 N) applied and the resulting displacement is measured for each. Higher force levels are also possible if required for more complete models. This data will then form the compliance curve for each subject at each measurement location (plotting displacement vs. force), from which a linear compliance number or nonlinear compliance function may be determined, as the case may be. These measurements may be repeated for all 3 Cartesian directions for the complete 3D compliance model.

Another challenge is measurement of shear compliances to complete the 3D model—the main question is whether to measure only at the surface or with some normal force into the abdomen. Normal compliances are easiest to measure physically in the lab. For shear compliances there is an additional challenge of ensuring that the probe does not slip during measurements. In general, the compliance of the measurement system should be at least an order of magnitude lower than that of the subject abdomen (two orders of magnitude was achieved for the back measurements, so this should be even better for the abdomen since the compliance of the abdomen is generally greater than that of the back).

Another application of the in vivo human tissue compliance modeling system is for determining heightened stiffness of muscle at tenderpoints in Fibromyalgia patients. Using the same basic methods outlined above, EMG leads were also connected to the subject. An expert subject performed various levels of voluntary contraction of muscles (in the lumbar, cervical, and trapezius regions, separately). The subject used the EMG display to hold various levels of voluntary contraction while the haptic device performed the compliance measurements (all while the subject held his breath).

Referring to FIG. 6, a sample data run is shown for the tenderpoint compliance measurement with voluntary muscle contraction (stiffening). FIG. 6 shows the raw displacement/time data for the lumbar region with 100 mV voluntary muscle contraction (artificial stiffness). A dynamic component can be seen in the displacement/time graphs of FIG. 6; the last data points in each case were used for the static compliance plots. That is, before the force was increased to the next step every 1.5 seconds, the final displacement was recorded as the correct one for the static compliance results. The subject on this particular day allowed significantly less displacement on the right side than the left, in the lumbar region.

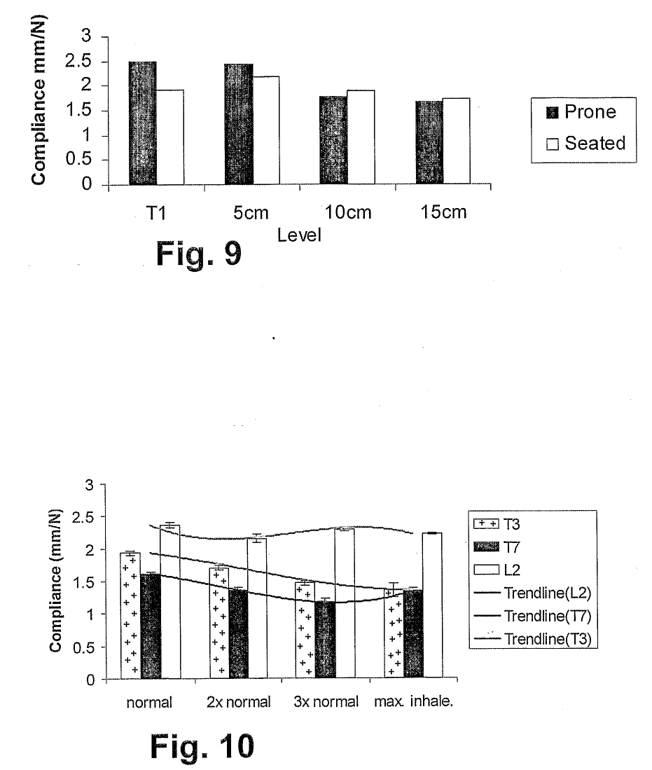

FIG. 7 shows the left and right compliance plots for the lumbar measurement region, for a voluntary contraction of 100 mV. It can be observed that data is nonlinear but may adequately be represented by a straight line fit in this force range (0.5-6 N). Though the displacements allowed in the subject's lumbar region were significantly different (see FIGS. 6 and 7 and note the y-intercepts of FIG. 7), the compliance, i.e. the slopes of the lines in FIG. 7, are similar: 1.35 mm/N for the right and 1.27 mm/N for the left.

From the calibration section the compliances of the measuring devices (PHANToM®3.0 haptic devices) were measured to be 0.4417 mm/N for the right device and 0.3748 mm/N for the left device, a fraction, perhaps significant, of the overall compliance measured in FIG. 7.

FIG. 8 summarizes the human lumbar measurement point (right and left) compliance data with voluntary contractions to create progressively stiffer tissue. In all cases it can be seen that increased voluntary contractions, leading to stiffer tissue, can indeed be measured by the system as increased stiffness (reduced compliance). Again, the subject was viewing the EMG readouts as a feedback mechanism to accurately effect voluntary muscle contractions. In FIG. 8, the percentage numbers indicated give the percent c vs. zero contraction.

An interesting consideration is how the compliance might change for seated vs. prone measurements of the same point. Two subjects were involved in this test. Eight (8) points (4 on the left and 4 on the right on the back) were tested on a subject. FIG. 9 shows the tissue compliance measured in the sitting and prone positions. In each posture two trials were implemented at each point. The average of these two results is shown as the compliance in FIG. 9.

Another interesting consideration is what effect thoracic volume has on the measured compliance. The subjects holds their breath during all statics and dynamic compliance measurements. To test the effect of how much breath is held (i.e. thoracic volume) on the resulting compliance measurement, the subject lay facedown on a table. He/she controlled the level of his/her breath by watching the scope. FIG. 10 shows that the subject's back compliance decreases with inhale increase. The tested compliance reaches the minimum value between 2× and 3× inhale. The compliance effects of thoracic volume varied between subjects, probably due to differences in gender, age, weight, and height etc.

As can be seen from the foregoing description, providing a method and apparatus for applying a predetermined force function to the surface of a test subject with a probe and measuring the displacement of the probe as a function of applied force facilitates measurement of tissue properties accurately and quickly, in vivo, in a non-invasive manner. Having described the invention in detail, those skilled in the art will appreciate that, given the present disclosure, modifications may be made to the invention without departing from the spirit of the inventive concept herein described. Therefore, it is not intended that the scope of the invention be limited to the specific and preferred embodiments illustrations as described. Rather, it is intended that the scope of the invention be determined by the appended claims. Furthermore, the preceding description is not meant to limit the scope of the invention. Rather, the scope of the invention is to be determined only by the appended claims and their equivalents.

1. A method that determines a model of a compliance related property of a target tissue of an animal or human test subject, the method comprising:
receiving a force function to be applied to the test subject;
applying a force that varies with time according to the force function on an exterior surface of the test subject that overlays the target tissue;
measuring a displacement of the probe during application of the force according to the force function; and
forming a compliance function that models the compliance related property by correlating the measured displacement to the applied force.

2. The method of claim 1 comprising positioning the probe such that the force is applied in a direction normal to the exterior surface.

3. The method of claim 1 wherein the step of applying the force function is performed by applying a series of applied force steps of increasing force and wherein the step of measuring the displacement is performed for each of the applied force steps.

4. The method of claim 3 wherein the step of measuring the displacement is performed at approximately an end of time duration of each applied force step.

5. The method of claim 1 wherein the step of forming a compliance function is performed by determining a best fit line that describes the displacement as a function of applied force and wherein the slope of the line is selected to model a compliance of the target tissue.

6. The method of claim 1 wherein the step of forming a compliance function is performed by determining a best fit curve that describes the displacement as a function of applied force and wherein the slope of the curve at each applied force is selected to model a compliance of the target tissue.

7. The method of claim 1 wherein the compliance related property is a viscous damping coefficient of the tissue, the method comprising:
determining a rate of change of displacement of the probe as a function of time; and
forming the compliance function using a model that correlates the rate of change of displacement to the force function.

8. The method of claim 7 wherein the step of forming a compliance function is performed by determining a first order linear model that expresses force as the sum of the product of the viscous damping coefficient and the first derivative of the displacement as a function of time and the product of a static spring coefficient and the displacement as a function of time.

9. The method of claim 7 wherein the step of forming a compliance function is performed by determining a second order linear model that expresses a change in displacement in response to an input force as a function of the first and second derivatives of the displacement as a function of time; a damping ratio, the natural frequency, and the displacement as a function of time.

10. The method of claim 1 wherein the step of applying the force is performed by applying a force that varies as a sinusoidal function.

11. The method of claim 1 further comprising monitoring EMG signals from sensors connected to the test subject and measuring displacement at predetermined EMG levels.

12. The method of claim 1 comprising repeating the measurement method periodically on a given subject to determine changes in tissue condition.

13. An apparatus that determines a model of a compliance related property of a target tissue in a test subject, the apparatus comprising:
a probe adapted to contact and apply force to an exterior surface of the test subject;
a probe driver that is adapted to receive a force function and cause the probe to apply a force that varies in time according to the force function and measure a displacement of the probe during application of the force;
a compliance modeler in communication with the probe driver that forms a compliance function that correlates measured displacement to the applied force; and
a compliance modeling interface that is configured to:
accept a force function from a user and transmit the force function to the probe driver;
receive displacement data from the probe driver; and
transmit the displacement data and data indicative of the force applied to the subject to the compliance modeler.

14. The apparatus of claim 13 wherein the probe driver is a haptic device that applies forces to the subject according to the force function received from the compliance modeling interface.

15. The apparatus of claim 13 comprising an EMG monitor that monitors and displays EMG level in the target tissue to test subject.

16. The apparatus of claim 13 wherein the probe driver positions the probe to contact the subject at a desired angle, wherein probe driver is configured to accept a value for the desired angle from the compliance modeling interface.

17. The apparatus of claim 13 comprising a user interface that provides an interface for a user to input a desired force function and displays the resulting compliance function to the user.

18. A method that determines a model of a compliance related property of a target tissue of an animal or human test subject, the method comprising:
receiving a force function to be applied to the test subject, wherein the force function is non-oscillating and varies with time;
with a haptic device, applying a non-oscillating force that varies with time according to the force function on an exterior surface of the test subject that overlays the target tissue;
with the haptic device, measuring a displacement of the probe during application of the force according to the non-oscillating force function; and
forming a compliance function that models the compliance related property by con-elating the measured displacement to the applied force.

19. The method of claim 18 wherein the step of applying the force function is performed by applying a series of applied force steps of increasing force and wherein the step of measuring the displacement is performed for each of the applied force steps.

20. The method of claim 19 wherein the step of measuring the displacement is performed at approximately an end of time duration of each applied force step.

21. The method of claim 18 wherein the step of forming a compliance function is performed by determining a best fit line that describes the displacement as a function of applied force and wherein the slope of the line is selected to model a compliance of the target tissue.

22. The method of claim 18 wherein the step of forming a compliance function is performed by determining a best fit curve that describes the displacement as a function of applied force and wherein the slope of the curve at each applied force is selected to model a compliance of the target tissue.

23. The method of claim 18 wherein the compliance related property is a viscous damping coefficient of the tissue, the method comprising:
determining a rate of change of displacement of the probe as a function of time; and
forming the compliance function using a model that correlates the rate of change of displacement to the force function.

24. The method of claim 23 wherein the step of forming a compliance function is performed by determining a first order linear model that expresses force as the sum of the product of the viscous damping coefficient and the first derivative of the displacement as a function of time and the product of a static spring coefficient and the displacement as a function of time.

25. The method of claim 23 wherein the step of forming a compliance function is performed by determining a second order linear model that expresses a change in displacement in response to an input force as a function of the first and second derivatives of the displacement as a function of time; a damping ratio, the natural frequency, and the displacement as a function of time.
